(19) United States
(12) Patent Application Publication
(10) Pub. No.: US 2026/0256410 A1
(43) Pub. Date: Sep. 3, 2026

(54) DIGITAL OLFACTORY

(71) Applicant: MOODIFY LTD., Kefar Sava (IL)

(72) Inventor: Idan AMIT, Ramat Gan (IL)

(21) Appl. No.: 19/165,020

(22) PCT Filed: Mar. 20, 2024
(86) PCT No.: PCT/IL2024/050287

§ 371 Date: Sep. 12, 2025

Related U.S. Application Data
(60) Provisional application No. 63/491,432, filed on Mar. 21, 2023.

(51) Int. Cl. A61B 5/00

(52) U.S. Cl. CPC A61B 5/4011; A61B 5/7203; A61B 5/7264

(57) ABSTRACT

There is presented a technique of providing a digital olfactory solution involving a quantified assessment of smells similarity and/or perceived intensity. The method includes processing an evaluation dataset resulting from performing direct distinction tests on a plurality of mixtures of a primary smell with a secondary smell added in different mixtures in different proportions; obtaining a tolerance factor characterizing the pair of the primary smell and the secondary smell, wherein the tolerance factor is indicative of the ratio of the secondary smell necessary to distinguish its mixture with the primary smell from the primary smell itself; and using the obtained tolerance factor as a quantified similarity parameter for enabling the digital olfactory solutions. The method can further include obtaining intensity factor being inversely proportional to the tolerance factor and being informative of the ratio of the smell of interest necessary to distinguish its presence in a mixture with odorless material.

### CROSS-REFERENCE TO RELATED APPLICATION

This application is a national phase filing under 35 C.F.R. § 371 of and claims priority to PCT Patent Application No. PCT/IL2024/050287, filed on Mar. 20, 2024, which claims the benefit of U.S. Provisional Application No. 63/491,432, filed on Mar. 21, 2023, the contents of which are hereby incorporated in their entireties by reference

### TECHNICAL FIELD

The presently disclosed subject matter relates to digital olfactory techniques and, more particularly, to digital olfactory techniques requiring a quantified similarity and/or perceptual intensity of smells.

### BACKGROUND

Digital olfactory technologies enable generating, transmitting, and receiving smell-enabled digital media usable for communication, gaming, virtual reality, extended reality, e-commerce, automotive and other applications. Likewise, digital olfactory is usable for a wide range of other applications, for example detection of diseases through breath, air quality surveillance, the food and beverage processing, fragrance engineering, etc.

Among the problems of digital olfactory are quantified and reproduceable methods for measuring smell likeness and differences.

Problems of smell distinction/similarity have been recognized in conventional art and various direct and indirect techniques have been developed to provide solutions.

It is noted that, in the description below, the terms similarity and distinctiveness (or alike) are used interchangeably as opposing terms. The distinctiveness of smells indicates how easily two or more odors can be set apart from each other, while the similarity of smells indicates the degree to which it is challenging to differentiate between two or more odors.

Direct methods of smell distinction are based on conducting experiments that measure distinction and/or similarity of smells.

For example, it is common to measure smell similarity by asking people to rank in a 0 to 100 scale how similar the smells are, asking whether two smells are distinct, asking to find the different smell between three smells, etc. One of the problems with such distinction tests is that they cannot quantify distinct yet close smells. Orange, lemon and gasoline are distinguishable, yet orange and lemon are closer to each other than to gasoline.

Indirect methods involve external sources of information for learning smells similarity (e.g. a perceptual database, chemical database, etc.). A common indirect similarity metrics family can be based on the application of general-purpose distance functions (e.g., Euclidean distance, cosine, Jaccard) to smell properties databases. Indirect methods can be based on semantics, chemical, biological properties, and/or perceptual properties. Among the problems of indirect methods is that they provide different values when using different databases, thereby reducing their reliability. Furthermore, the indirect metrics should be evaluated and learned by fitting to direct perceptual results.

In general, direct methods can be more accurate since they are based on a specific pair evaluation and provide information on that specific pair of smells. Indirect methods are more scalable, requiring a use of O(n) smells instead of O(n²) pairs. In the smell research domain, direct similarity methods are usually considered the ‘ground-truth’ of smell similarity and are therefore used to evaluate the performance and develop novel indirect similarity methods.

The article by M. J. Olsson and W. S. Cain. (M. J. Olsson and W. S. Cain Psychometrics of odor quality discrimination: method for threshold determination. Chemical senses, 25 (5): 493-499, 2000) discloses a method (referred to hereinafter as SURE (substitution-reciprocity) method) of studying smell similarity using binary mixtures taken in various proportions. The SURE method enables comparison stimuli at about the same level of perceived intensity and causes a difference only in odor quality. Thresholds for both directions (from A to B and from B to A) are averaged, leading to a symmetric function.

A review of the smell distinction methods known in contemporary art can be found in the article of Wise et al. (P. M. Wise, M. J. Olsson, and W. S. Cain. Quantification of odor quality. Chemical senses, 25 (4): 429-443, 2000).

The cited references herein teach background information that may be applicable to the presently disclosed subject matter. Therefore, the full contents of these publications are incorporated by reference herein where appropriate for appropriate teachings of additional or alternative details, features and/or technical background.

### GENERAL DESCRIPTION

The inventor has recognized that quantification of similarity between smells requires considering the non-symmetric nature of smell distinguishability in smell mixtures. The typical belief that similarity indicates symmetry, meaning that if A is similar to B, then B is similar to A to the same extent, does not hold true for smell mixtures (unless there have been implemented specific measures to equalize perceived intensity). Mixing a high-intensity secondary smell into a low-intensity primary smell becomes perceptible in a small ratio, but achieving noticeability of adding a low-intensity secondary smell necessitates its higher ration in the mixture. For example, a mixture resulting from a few drops of gasoline (secondary smell) added to a large amount of orange oil (primary smell) will be easily distinguished from the pure orange oil smell. However, a significantly larger quantity of lemon oil (secondary smell) must be added to attain a mixture with the same distinctiveness from the pure orange oil smell.

Accordingly, the inventor has appreciated that there is a need to provide a quantified characteristic of similarity of two smells without mitigating the influence of the intensity on their distinguishability. Such characteristic is referred to hereinafter as a “tolerance factor”. Tolerance factor is indicative of the ratio of a secondary smell necessary to distinguish its mixture with a primary smell from the primary smell itself.

Low values of the tolerance factor are indicative of high distinctiveness (low similarities) between the smells and high values of the tolerance factor are indicative of low distinctiveness (high similarities) of the smells in the pair. Accordingly, the tolerance factor presents the non-symmetric distinctiveness of smells in the pairs (e.g. respective tolerance factors can present the low tolerance of lemon to gasoline and high tolerance of gasoline to lemon).

Thus, the tolerance factor is a quantified metric usable for digital olfactory solutions involving computerized assessment of smells similarity/distinctiveness.

It is noted that unless specifically stated otherwise, the term “digital olfactory solution” used herein should be expansively construed to cover any technique dealing with turning the smells into digital data with further ability of distributing, storing, analyzing the resulted data, and restituting the digital data back into the smells when necessary.

It is further noted that unless specifically stated otherwise, the terms “mixture of smells” or alike used herein should be expansively construed to cover a combination of individual material sources of different smells that are perceived simultaneously. The material sources can be combined and, accordingly, the smells in the mixture can origin from a single mixed source. Alternatively, the smells can origin from unmixed material sources (e.g. from a plurality of diffusers, bottles with different materials, etc.) spreading respective smells for simultaneous perception. Likewise, the term “ratio of a smell in a mixture” refers to a ratio of a respective material source.

In accordance with certain aspects of the presently disclosed subject matter there is provided a method of providing a digital olfactory solution involving a quantified assessment of smells similarity. The method comprises: processing, by a computer, an evaluation dataset resulting from performing direct distinction tests on a plurality of mixtures of a first material source of a primary smell with a second material source of a secondary smell added in different mixtures in different proportions; obtaining, by the computer, a tolerance factor characterizing a pair of the primary smell and the secondary smell, wherein the tolerance factor is indicative of the ratio of the second material source of the secondary smell necessary to distinguish the smell of its mixture with the first material source of the primary smell from the primary smell itself; and using the obtained tolerance factor as a quantified similarity parameter for enabling the digital olfactory solutions.

By way of non-limiting examples, the tolerance factor can be used for at least one of: a) in smell communication, ensuring the smells similarity between a smell at a receiver end and a corresponding smell at a transmitter end; b) assessing the smells similarity between a sampled smell and a reference smell associated with spoilage, pollution or hazardous materials; c) assessing smell similarity between an original smell and an altered smell in a smell reformulation process.

In accordance with further aspects and, optionally, in combination with other aspects of the presently disclosed subject matter, the tolerance factor can be obtained in consideration of probabilities of classifications errors.

In accordance with further aspects and, optionally, in combination with other aspects of the presently disclosed subject matter, the evaluation dataset can be processed by applying a parametric model and the tolerance factor can be calculated as the optimal value of a smell distinctiveness. For example, the tolerance factor is calculated as a Maximum Likelihood Estimation best explaining the observations.

The inputs of the parametric model can comprise a ratio of the secondary smell in the mixture and a self-confusion characteristic of the primary smell, and the objective of applying the parametric model to the evaluation dataset can be finding the optimal value of the tolerance factor as a smell distinction parameter.

In accordance with further aspects and, optionally, in combination with other aspects of the presently disclosed subject matter, the primary smell can be used as a reference smell and the secondary smell is used as a sampled smell, the method further comprises: comparing the obtained tolerance factor characterizing the pair of the reference smell and the sampled smells with a predefined threshold tolerance value associated with the reference smell; and assessing the sample smell and the reference smell as being similar when the obtained tolerance factor is above the threshold tolerance value.

In accordance with further aspects and, optionally, in combination with other aspects of the presently disclosed subject matter, the first material source of the primary smell can be an odorless material, the method further comprises: using the obtained tolerance factor as an intensity factor being inversely related to the obtained tolerance factor and informative of the ratio of the second material source of the secondary smell necessary to distinguish its presence in the mixture; and using the intensity factor as a distinctiveness thresholds informative of perceived intensity of the secondary smell.

The method can further comprise: obtaining intensity factors for a plurality of smells; generating an intensity scale for the plurality of smells, wherein the intensity scale is configured to represent the respective distinctiveness thresholds as a function of blending the odorless material source, thereby enabling comparing perceived intensity of different smells from the plurality of smells.

In accordance with other aspects and, optionally, in combination with the above aspects of the presently disclosed subject matter, there is provided a method of providing a digital olfactory solution involving a quantified assessment of a perceived smell intensity. The method comprises: processing, by a computer, an evaluation dataset resulting from performing direct distinction tests on a plurality of mixtures of an odorless material source of a primary smell with a second material source of a smell of interest added in different mixtures in different proportions; obtaining, by the computer, an intensity factor informative of the ratio of the second material source of the smell of interest necessary to distinguish the smell's presence in the mixture; and using the obtained intensity factor as a distinctiveness threshold for enabling the digital olfactory solutions.

The method can further comprise: obtaining intensity factors for a plurality of smells; generating an intensity scale for the plurality of smells, wherein the intensity scale is configured to represent the respective distinctiveness thresholds as a function of blending the odorless material source, thereby enabling comparing perceived intensity of different smells from the plurality of smells.

In accordance with other aspects of the presently disclosed subject matter, there is provided a computing system configured to perform the operations of the methods above.

In accordance with other aspects of the presently disclosed subject matter, there is provided a non-transitory computer-readable medium comprising instructions that, when executed by a computing system comprising a memory storing a plurality of program components executable by the computing system, cause the computing system to operate in accordance with the methods above.

### BRIEF DESCRIPTION OF THE DRAWINGS

In order to understand the presently disclosed subject matter and to see how it can be carried out in practice, embodiments will be described, by way of non-limiting examples, with reference to the accompanying drawings, in which:

FIG. 1 illustrates a generalized flow-chart of a method of providing a digital olfactory solution in accordance with certain embodiments of the presently disclosed subject matter;

FIG. 2 illustrates a generalized flow-chart of obtaining a tolerance factor characterizing a pair of smells in accordance with certain embodiments of the presently disclosed subject matter;

FIG. 3 illustrates a generalized flow-chart of non-limiting examples of using the tolerance factor in digital olfactory solutions in accordance with certain embodiments of the presently disclosed subject matter;

FIG. 4 illustrates a generalized flow-chart of obtaining a perceived intensity factor in accordance with certain embodiments of the presently disclosed subject matter; and

FIG. 5 illustrates a generalized block diagram of a computerized system capable of smell similarity quantification in accordance with certain embodiments of the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “applying”, “comparing”, “assessing”, “matching” or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the computerized system and processing and memory (PMC) circuitry therein disclosed in the present application.

The operations in accordance with the teachings herein may be performed by a computer specially constructed for the desired purposes or by a general-purpose computer specially configured for the desired purpose by a computer program stored in a non-transitory computer-readable storage medium.

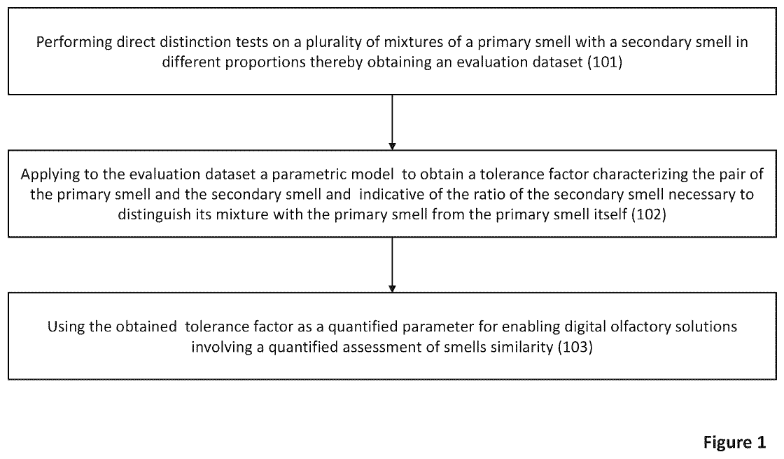

Bearing this in mind, attention is drawn to FIG. 1 illustrating a generalized flow-chart of a method of providing a digital olfactory solution in accordance with certain embodiments of the presently disclosed subject matter.

The method comprises performing direct distinction tests on a plurality of mixtures of a primary smell with a secondary smell added in different proportions (i.e. the ratio of the secondary smell in each mixture varies across the plurality of mixtures), thereby obtaining (101) an evaluation dataset. The dataset comprises observations of distinctiveness between the mixtures and the primary smell.

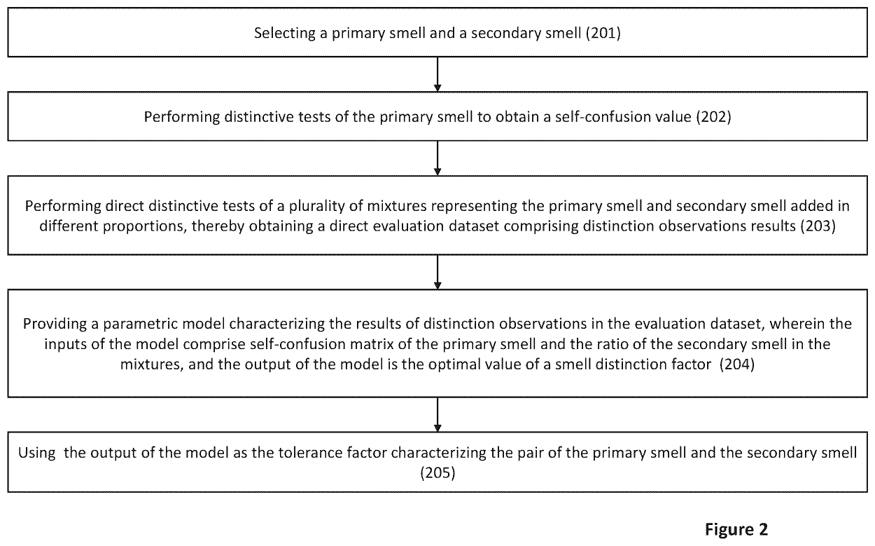

As will be further detailed with reference to FIG. 2, a computer processes the evaluation dataset to obtain (102) a tolerance factor characterizing the pair of primary and secondary smells. The tolerance factor is indicative of the ratio of the secondary smell necessary to distinguish its mixture with the primary smell from the primary smell itself. The dataset can be processed by applying a parametric model and calculating the tolerance factor as the optimal value of a smell distinctiveness (e.g. as a Maximum Likelihood Estimation best explaining the observations).

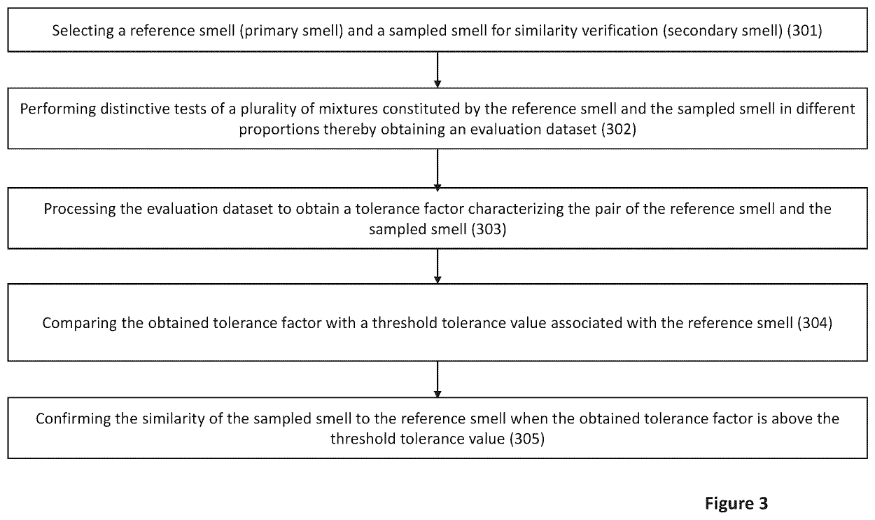

As further detailed with reference to FIG. 3, the obtained tolerance factor is usable as a quantified similarity parameter for enabling (103) digital olfactory solutions involving a quantified assessment of smells similarity.

Referring to FIG. 2, there is illustrated a generalized flow-chart of obtaining a tolerance factor characterizing a pair of smells in accordance with certain embodiments of the presently disclosed subject matter.

The method includes obtaining data informative of a selected (201) primary smell and secondary smell and of a self-confusion value (202) of the primary smell. The self-confusion value results from distinctive tests performed for the primary smell. By way of non-limiting example, such value can be calculated as accuracy score metric usable in binary classification One can also give different weight to false positives and false negative, balancing them as in parametrize F1.

The method further includes performing direct distinctive tests of a plurality of mixtures representing the primary smell and secondary smell added in different proportions. As a result of the distinctive tests, there is obtained (203) a direct evaluation dataset comprising distinction observations.

Direct distinction tests can be provided by any suitable method, some of them are known in the art. By way of non-limiting example, the tests can be provided with the help of pairs perceived similarity tests, pairs same/different classification, and/or triplet “Find the different” tests disclosed in the article by A. Ravia et. al (A. Ravia, K. Snitz, D. Honigstein, M. Finkel, R. Zirler, O. Perl, L. Secundo, C. Laudamiel, D. Harel, and N. Sobel. A measure of smell enables the creation of olfactory metamers. Nature, 588 (7836): 118-123, 2020).

Optionally, the set of mixtures required for direct distinct evaluation tests can be prepared using a brute force approach, i.e. given a desired step resolution, create all mixtures of the material sources of smells A and B (e.g., for a step resolution of 5%, the mixtures are: pure A, 95% A and 5% B, 90% A and 10% B, . . . , pure B).

Going over all mixtures in brute force will have O(n) complexity (of time and samples). In certain embodiments the complexity can be reduced due to monotonicity assumption—the higher the ratio of the material source of the secondary smell, the higher the distinction. Assuming monotonicity allows more efficient search algorithms. For example, by using a binary search, one can improve the complexity to O(log(n)) while an interpolation search can improve the complexity to O(log(log(n))).

The direct evaluation dataset comprises data informative of result j(x) obtained in observations o₁ . . . oₙ of different people trying to distinguish different mixtures m(x). For example, in certain embodiments, a person can be asked to distinguish between the mixture, m(x) and the primary smell. The result j(x) of such distinction observation can be a binary judgement informative if the person considered m(x) to be similar to the primary smell. A positive judgment claims that the smells are distinct, and a negative judgment claims that they are similar.

The evaluation dataset is processed by a computer to provide (204) a parametric model characterizing the results of the distinction observations in the evaluation dataset. The provided parametric model comprises a parametric family in which the distinction is due to a ratio of the secondary smell in the mixture and a tolerance factor of the pair of the primary smell and the secondary smell. Optionally, to enhance precision and resilience against noise, the parametric family can incorporate a self-confusion characteristic of the primary smell.

The inputs of the model include the ratio of the secondary smell in the mixtures and the self-confusion characteristic of the primary smell. The objective of applying the parametric model to the evaluation dataset is to find the optimal value of a smell distinction parameter distinction (primary, secondary). Such output of the model can be used (205) as the tolerance factor characterizing the pair of the primary smell and the secondary smell.

In accordance with certain embodiments of the presently disclosed subject matter, the output of the model (and, accordingly, the tolerance factor) can be defined as a value of distinction parameter distinction (primary, secondary) maximizing the likelihood (MLE) of the observations. Techniques of applying the MLE (Maximal Likelihood Estimation) approach to observation results are detailed, for example, in the article of A. P. Dawid and A. M. Skene (A. P. Dawid and A. M. Skene. Maximum likelihood estimation of observer error-rates using the EM algorithm. Journal of the Royal Statistical Society. Series C (Applied Statistics), 28 (1): 20-28, 1979) incorporated herein by reference.

The Maximum Likelihood Estimation (MLE) finds the most likely parameter values explaining the set of observations.

The probability of distinction of one observation is dis(oᵢ)

$$dis ( {o}_{i} ) = m ( {o}_{i} ) [ secondary ] \cdot distinction ( primary , secondary ) + m ( {o}_{i} ) [ primary ] \cdot confusion ( primary , primary ) \qquad ( 1 )$$

A mixture mixture (primary₁₋ₓ, secondaryₓ) is a binary mixture with 1-x percents primary and x percents secondary. Put formally,

$$mixture ( {primary}_{1 - x} , {secondary}_{x} ) \text{} contains ( {primary}_{1 - x} , {secondary}_{x} ) [ primary ] = 1 - x \qquad ( 2 )$$

$$primary smell and ( {primary}_{1 - x} , {secondary}_{x} ) [ secondary ] = x \qquad ( 3 )$$

$$secondary smell .$$

Hence for a mixture (primary₁₋ₓ, secondaryₓ), the value of Equation (1) is

$$( 1 - x ) distinction ( primary , secondary ) + x \cdot confusion ( primary , primary ) \qquad ( 4 )$$

In certain embodiments, the probability of a positive judgment P(j(oᵢ)|dis(oᵢ)) of a single observation oᵢ can be evaluated using a model in which dis(oᵢ) is the probability of positive judgment and 1−dis(oᵢ) is the probability for a negative judgment. In other embodiments, the probability can be evaluated in more sophisticated ways for considering the participant distinction performance.

Given the set of observations o₁, . . . , oₙ, the likelihood of all judgments is

$$arg {max}_{x} \prod P ( j ( {o}_{i} \vert dis ( {o}_{i} ) ) \qquad ( 5 )$$

where x is our distinction (primary, secondary), used in dis(oᵢ) given in Equation (1).

Thus, using the maximum likelihood estimation method allows to find the value of tolerance factor as best explaining the observations.

It is noted that the noise can be further reduced by incorporating a confusion matrix into the classification, thereby mitigating the noise caused by people's failures in identifying distinct smells or confusing the same smell to be distinct.

For example, using a confusion matrix for evaluating classifier performance in a manner detailed in the incorporated herein by reference article by I. Amit and D. G. Feitelson (I. Amit and D. G. Feitelson. Corrective commit probability: a measure of the effort invested in bug fixing. Software Quality Journal, pages 1-45, August 2021. I. Amit and D. G. Feitelson) can be helpful to adapt the result to achieve maximum likelihood predictions. This approach can be applied to the entire population of subjects. Given enough data on subjects (either based on performance in previous olfactory tests, or other characteristics), one can adapt the results at the subject level, thereby reducing the noise caused by the individual differences.

Referring to FIG. 3, there is illustrated a generalized flow-chart of a non-limiting example of using the tolerance factor in digital olfactory solutions in accordance with certain embodiments of the presently disclosed subject matter.

Many digital olfactory solutions necessitate assessing similarity between a reference smell and a sampled smell to be verified.

By way of non-limiting example, in smell communication assessing smells similarity can ensure that a smell at a receiver end (sampled smell) is similar to a corresponding smell at a transmitter end (reference smell).

By way of another non-limiting example, detecting the smells similarity between a sampled smell and a reference smell associated with spoilage, pollution, hazardous materials or alike can be helpful in the control processes, whether in food production, environmental monitoring, health care, etc.

By way of yet another non-limiting example, assessing smell similarity is an important part of product development in cosmetics, fragrance and perfume industries. Likewise, assessing smell similarity is the essential part in smell reformulation processes requiring changing the composition of smell ingredients whilst keeping similarity of the original and the altered smells.

As illustrated in FIG. 3, upon selecting (301) a reference smell and a smell for similarity verification, there is obtained (302) the evaluation dataset. The evaluation dataset is obtained by performing distinctive tests of a plurality of mixtures constituted by the reference smell (primary smell) and the sampled smell (secondary smell) in different proportions. It is noted that the distinctive tests are provided without mitigating the influence of smells' intensity on their distinguishability.

The method further includes processing the evaluation dataset to obtain (303) a tolerance factor characterizing the pair of the reference and the sampled smells. The obtained tolerance factor is compared (304) with a predefined threshold tolerance value associated with the reference smell (e.g. a self-confusion value of the reference smell).

The sampled smell is confirmed (305) as similar to the reference smell when the obtained tolerance factor is above the threshold tolerance value.

Thus, the teachings of the presently disclosed subject matter enable providing a quantified similarity assessment of two smells whilst acknowledging their intensity.

By way of another non-limiting example, the teachings of the presently disclosed subject matter can enable quantified description of a smell space.

It is noted that direct methods require preparing the smells in a lab and running distinction experiments. They consume significant resources and time, resulting in a limited number of observations and labels related to smells. Machine learning prediction in lack of labels can be provided with the help of weak supervision learning. Weak supervision learning models can be applied by using direct evaluations as labels and building and evaluating indirect methods as models.

Given a perceptual or chemical dataset, one can build a smell similarity distance metric by applying a general-purpose distance metric such as Euclidean distance or Jaccard. However, the indirect metrics need to be evaluated and learned by fitting to direct perceptual results. In order to know which result fit human perception better, these metrics should be evaluated on direct perception results disclosed in, as obtained from our method. Since direct datasets are usually small, one can use weakly supervised methods to calibrate and aggregate distance functions. Non-limiting examples of such calibration and aggregation are disclosed in the US Patent Publication No. US2019/0164086.

Most datasets of smell description are based on structured linguistic description (e.g, ‘nutty’, ‘fruity’) or on ranking of such features (e.g., 3 in ‘nutty’, 5 in ‘fruity’) (see, for example, C. The Good Scents. Flavor, fragrance, food and cosmetics ingredients information, 2023). To reliably use these datasets, one needs to possess the ability to identify informative features, agree on their meaning, and label them correctly and consistently. Furthermore, given such a description, it is hard to understand the described entity (e.g., understand that an apple pie is described).

The tolerance factor enables describing the entities based on their similarity (e.g., the smells of orange and lemon are close). One can apply similarity-based algorithms, such as k-nearest neighbor, and obtain a robust description based on relations without the need to fix and name the features.

A dataset of tolerance factors between smell pairs can be useful for training a mathematical learning model capable of predicting smell similarities. As detailed above, the tolerance factor is defined using the probability of classifications errors. Accordingly, in order to fit the perceptual evaluations, a similarity prediction model shall consider the probabilistic behavior of tolerance. In accordance with certain embodiments, the loss function is trained in a probabilistic manner. For small numbers of samples, it is computed, using binomial distribution, the probability of getting k classification errors out of n classifications, given the tolerance probability. For a larger number of samples, one can use the Gaussian distribution as an approximation. Such probabilistic loss function is further used to build the similarity prediction model on a similarity dataset (e.g., perceptual, chemical, etc.).

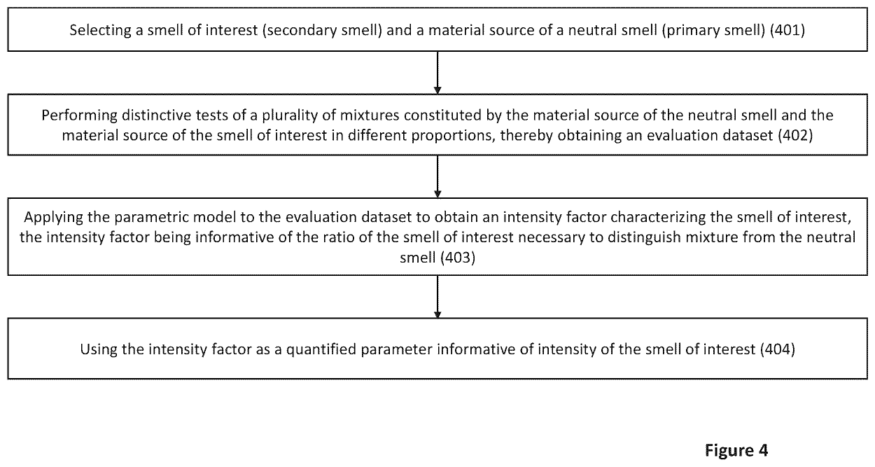

Referring to FIG. 4, there is illustrated a quantified assessment of a smell perceived intensity in accordance with the teachings of the presently disclosed subject matter. The method starts with selecting (401) a smell of interest as a secondary smell. An odorless material is selected as a source of a neutral primary smell. The mixtures can be constituted by the odorless solvent (e.g., Water (CAS 7732-18-5), Ethanol (CAS 64-17-5), DPG (di-propylene glycol, CAS 25265-71-8), PG (DL-1,2-propanediol, CAS 57-55-6), IPM (isopropyl myristate, CAS 110-27-0), etc.) and the material source of the smell of interest added in different proportions. Performing distinctive tests over a plurality of such mixtures provides (402) an evaluation dataset.

The method further includes applying the parametric model to the evaluation dataset to obtain (403) the tolerance factor. Such tolerance factor obtained for the mixtures with the neutral smell can characterize the perceived intensity of the smell of interest. The intensity factor is inversely proportional to the tolerant factor and is informative of the ratio of the smell of interest necessary to distinguish its presence in the mixture (i.e. distinguish its mixture with the neutral smell from the neutral smell itself). Such intensity factor can be used (404) as a quantified parameter (distinctiveness threshold) informative of perceived intensity of the smell of interest.

In accordance with certain embodiments of the presently disclosed subject matter, quantification of intensity further allows to build an intensity scale. The intensity scale represents the distinctiveness thresholds as a function of blending an odorless material source, thereby enabling comparing perceived intensity of different smells.

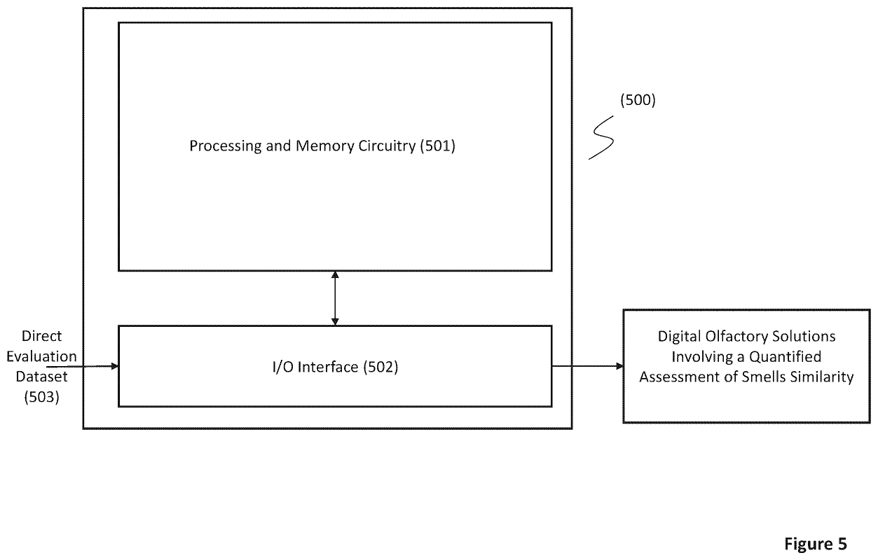

FIG. 5 illustrates a generalized block diagram of a computerized system capable of smell similarity quantification in accordance with certain embodiments of the presently disclosed subject matter. The illustrated system 500 comprises input/output interface 502 operatively connected processing and memory circuitry (PMC) 501 comprising a processor and a memory (not shown separately within the PMC).

The system is configured to receive direct evaluation dataset 503 via input/output interface 502. PMC 501 is configured to execute computer-readable instructions implemented on a non-transitory computer-readable storage medium. The instructions, when executed by PMC 501 cause the computing system to process the received evaluation dataset 503 and enable quantification of similarity and/or intensity of smells as detailed with reference to FIGS. 1-4.

The computerized system 500 can be a standalone entity, or can be integrated, fully or partly, with other systems.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the presently disclosed subject matter may be, at least partly, implemented on a suitably programmed computer. Likewise, the presently disclosed subject matter contemplates a computer program being readable by a computer for executing the method of the presently disclosed subject matter. The presently disclosed subject matter further contemplates a non-transitory computer-readable memory tangibly embodying a program of instructions executable by the computer for executing the method of the presently disclosed subject matter.

Those skilled in the art will readily appreciate that various modifications and changes can be applied to the embodiments of the presently disclosed subject matter as hereinbefore described without departing from its scope, defined in and by the appended claims.

1. A method of providing a digital olfactory solution involving a quantified assessment of smells similarity, the method comprising:
processing, by a computer, an evaluation dataset resulting from performing direct distinction tests on a plurality of mixtures of a first material source of a primary smell with a second material source of a secondary smell added in different mixtures in different proportions;
obtaining, by the computer, a tolerance factor characterizing a pair of the primary smell and the secondary smell, wherein the tolerance factor is indicative of the proportion of the second material source required for its mixture with the first material source to be distinguishable from the primary smell alone; and
using the obtained tolerance factor as a quantified similarity parameter for enabling the digital olfactory solutions.

2. The method of claim 1, wherein the tolerance factor is used for at least one of:
a. in smell communication, ensuring the smells similarity between a smell at a receiver end and a corresponding smell at a transmitter end;
b. assessing the smells similarity between a sampled smell and a reference smell associated with spoilage, pollution or hazardous materials;
c. assessing smell similarity between an original smell and an altered smell in a smell reformulation process.

3. The method of claim 1, wherein the primary smell is used as a reference smell and the secondary smell is used as a sampled smell, the method further comprising:
comparing the obtained tolerance factor characterizing the pair of the reference smell and the sampled smells with a predefined threshold tolerance value associated with the reference smell; and
assessing the sample smell and the reference smell as being similar when the obtained tolerance factor is above the threshold tolerance value.

4. The method of claim 1, wherein the tolerance factor is obtained in consideration of probabilities of classifications errors.

5. The method of claim 1, wherein the evaluation dataset is processed by applying a parametric model and the tolerance factor is calculated as the optimal value of a smell distinctiveness.

6. The method of claim 5, wherein the tolerance factor is calculated as a Maximum Likelihood Estimation best explaining the observations.

7. The method of claim 5, wherein inputs of the parametric model comprise a ratio of the secondary smell in the mixture and a self-confusion characteristic of the primary smell, and the objective of applying the parametric model to the evaluation dataset is to find the optimal value of the tolerance factor as a smell distinction parameter.

8. The method of claim 1, wherein the first material source of the primary smell is an odourless material, the method further comprising:
using the obtained tolerance factor as an intensity factor being inversely related to the obtained tolerance factor and informative of the ratio of the second material source of the secondary smell necessary to distinguish its presence in the mixture; and
using the intensity factor as a distinctiveness thresholds informative of perceived intensity of the secondary smell.

9. The method of claim 8 further comprising:
obtaining intensity factors for a plurality of smells;
generating an intensity scale for the plurality of smells, wherein the intensity scale is configured to represent the respective distinctiveness thresholds as a function of blending the odourless material source, thereby enabling comparing perceived intensity of different smells from the plurality of smells.

10. A method of providing a digital olfactory solution involving a quantified assessment of a perceived smell intensity, the method comprising:
processing, by a computer, an evaluation dataset resulting from performing direct distinction tests on a plurality of mixtures of an odorless material source of a primary smell with a second material source of a smell of interest added in different mixtures in different proportions;
obtaining, by the computer, an intensity factor informative of the ratio of the second material source of the smell of interest necessary to distinguish the smell's presence in the mixture; and
using the obtained intensity factor as a distinctiveness threshold for enabling the digital olfactory solutions.

11. The method of claim 10 further comprising:
obtaining intensity factors for a plurality of smells;
generating an intensity scale for the plurality of smells, wherein the intensity scale is configured to represent the respective distinctiveness thresholds as a function of blending the odourless material source, thereby enabling comparing perceived intensity of different smells from the plurality of smells.

12. A computing system configured to provide a digital olfactory solution involving a quantified assessment of smells similarity, the system comprising a computer configured to perform the operations comprising:
processing computer, an evaluation dataset resulting from performing direct distinction tests on a plurality of mixtures of a first material source of a primary smell with a second material source of a secondary smell added in different mixtures in different proportions;
obtaining a tolerance factor characterizing a pair of the primary smell and the secondary smell, wherein the tolerance factor is indicative of the proportion of the second material source required for its mixture with the first material source to be distinguishable from the primary smell alone; and
using the obtained tolerance factor as a quantified similarity parameter for enabling the digital olfactory solution.

13. The system of claim 12, wherein the tolerance factor is used for at least one of:
a. in smell communication, ensuring the smells similarity between a smell at a receiver end and a corresponding smell at a transmitter end;
b. assessing the smells similarity between a sampled smell and a reference smell associated with spoilage, pollution or hazardous materials;
c. assessing smell similarity between an original smell and an altered smell in a smell reformulation process.

14. The system of claim 12, wherein the primary smell is used as a reference smell and the secondary smell is used as a sampled smell, the method further comprising:
comparing the obtained tolerance factor characterizing the pair of the reference smell and the sampled smells with a predefined threshold tolerance value associated with the reference smell; and
assessing the sample smell and the reference smell as being similar when the obtained tolerance factor is above the threshold tolerance value.

15. The system of claim 12, wherein the evaluation dataset is processed by applying a parametric model and the tolerance factor is calculated as the optimal value of a smell distinctiveness.

16. The system of claim 15, wherein the tolerance factor is calculated as a Maximum Likelihood Estimation best explaining the observations.

17. The system of claim 15, wherein inputs of the parametric model comprise a ratio of the secondary smell in the mixture and a self-confusion characteristic of the primary smell, and the objective of applying the parametric model to the evaluation dataset is to find the optimal value of the tolerance factor as a smell distinction parameter.

18. The system of claim 12, wherein the first material source of the primary smell is an odourless material, the method further comprising:
using the obtained tolerance factor as an intensity factor being inversely related to the obtained tolerance factor and informative of the ratio of the second material source of the secondary smell necessary to distinguish its presence in the mixture; and
using the intensity factor as a distinctiveness thresholds informative of perceived intensity of the secondary smell.

19. The system of claim 18 further comprising:
obtaining intensity factors for a plurality of smells;
generating an intensity scale for the plurality of smells, wherein the intensity scale is configured to represent the respective distinctiveness thresholds as a function of blending the odourless material source, thereby enabling comparing perceived intensity of different smells from the plurality of smells.

20. A non-transitory computer-readable medium comprising instructions that, when executed by a computing system comprising a memory storing a plurality of program components executable by the computing system, cause the computing system to providing a digital olfactory solution involving a quantified assessment of smells similarity, providing the digital olfactory solution comprising:
processing an evaluation dataset resulting from performing direct distinction tests on a plurality of mixtures of a first material source of a primary smell with a second material source of a secondary smell added in different mixtures in different proportions;
obtaining a tolerance factor characterizing a pair of the primary smell and the secondary smell,
wherein the tolerance factor is indicative of the proportion of the second material source required for its mixture with the first material source to be distinguishable from the primary smell alone; and
using the obtain tolerance factor as a quantified similarity parameter for enabling the digital olfactory solutions.
